(19) United States
(12) Patent Application Publication
(10) Pub. No.: US 2025/0391506 A1
(43) Pub. Date: Dec. 25, 2025

(54) DNA Sequencing Using Viterbi-Like Correlation Analysis

(71) Applicant: Western Digital Technologies, Inc., San Jose, CA (US)

(72) Inventors: Iouri Oboukhov, Rochester, MN (US); Niranjay Ravindran, Rochester, MN (US); Richard Galbraith, Rochester, MN (US); Austin Striegel, Rochester, MN (US)

(21) Appl. No.: 18/753,421

(22) Filed: Jun. 25, 2024

(51) Int. Cl. G16B 30/00; G16B 40/00

(52) U.S. Cl. CPC G16B 30/00; G16B 40/00

(57) ABSTRACT

Example systems and methods for de novo sequencing of DNA or DNA-like sequences using Viterbi-like correlation analysis are described. A sequencing system receives the read data for multiple copies of a DNA strand from a sequence reader, such as a nanopore reader. The sequencing system generates a convolutional matrix based on one copy and a reference matrix based on another copy and uses them to generate a correlation matrix. A most likely path through the correlation matrix is determined to identify and correct errors between the two copies.

### TECHNICAL FIELD

The present disclosure relates to deoxyribonucleic acid (DNA) sequencing and sequencing of other genomic or DNA-like (e.g., nucleic acid based) sequences. In particular, the present disclosure relates to addressing errors in de novo sequencing of DNA.

### BACKGROUND

De novo gene sequencing involves sequencing novel genomes or genomic data where no reference sequence is available for alignment. For example, sequence reads from an unknown source of DNA or another DNA-like (e.g., nucleic acid (NA) based) substance, such as ribonucleic acid (RNA), may be assembled as contiguous sequences based on the output of a nanopore reader or base sensing device that converts an electrical signal across the NA-based strand into sequence data for further processing (NA-based strands will be referred to as DNA strands for simplicity of description below, but the description is not intended to be limiting to DNA applications). The DNA may be amplified through polymerase chain reaction (PCR) technology such that there are multiple copies of the DNA strands, resulting in multiple copies of the sequence data. Due to the nature of DNA and amplification processes, errors will occur in the redundant copies of the “same” DNA sequence. Additional data processing may be applied to the read data from the multiple copies of the strand to identify and correct errors across copies and achieve a “true” copy of the DNA sequence. The longer the DNA strands are, the more data processing may be required to correct the errors. In some applications, the goal of de novo genomic sequencing is to generate a “true” reference genomic sequence to improve the efficiency of further sequencing using draft quality, reference quality, and/or reference aligned sequencing methods.

There is a need for technology that applies more efficient error correction to de novo sequencing of DNA strands and, more particularly, more efficient identification and correction of insertion and deletion errors that represent small (but relatively common) shifts in the DNA sequences may be needed.

### BRIEF DESCRIPTION OF THE DRAWINGS

The techniques introduced herein are illustrated by way of example, and not by way of limitation, in the figures of the accompanying drawings in which like reference numerals are used to refer to similar elements.

FIG. 1A is a block diagram of a prior art de novo DNA sequencing process

FIG. 1B is a block diagram of a prior art DNA data storage process.

FIG. 1C is a block diagram of a prior art DNA data storage decoding process for oligos encoded with binary data.

FIG. 2A is a block diagram of an example de novo sequencing system using Viterbi-like processing of a correlation matrix for correcting insertions and deletions.

FIG. 2B is a block diagram of an example encoding system and example decoding system for DNA data storage using Viterbi-like processing of a correlation matrix for correcting insertions and deletions.

FIGS. 3A and 3B are diagrams of oligo data processing to correct for insertions and deletions prior to applying ECC processing.

FIG. 4A is a block diagram of an oligo data processing system using Viterbi-like processing of a correlation matrix to determine data offsets from insertions and deletions.

FIG. 4B includes example matrix diagrams showing determination of data offsets from the correlation matrix processing of FIG. 4A.

FIG. 5 is an example method for correcting insertions and deletions based on Viterbi processing of a correlation matrix, such as using the decoding systems of FIGS. 2B and 4A.

FIG. 6 is an example method for correcting oligo read data based on processing multiple copy pairs of oligo read data, such as using the decoding systems of FIGS. 2B and 4A.

FIG. 7 is an example method for determining probabilities of data shifts using a Viterbi algorithm, such as using the decoding systems of FIG. 2B and 4A.

### SUMMARY

Various aspects for using a correlation matrix and Viterbi algorithm for correcting errors during de novo sequencing of genomic data or as part of recovering sequences used in DNA-based data storage.

One general aspect includes a system that includes a sequencer configured to: receive read data determined from sequencing at least two copies of a strand, where the strand may include a sequence of bases or base pairs; determine a convolutional matrix based on a first copy of the strand, where each column of the convolutional matrix corresponds to a base pair offset of the first copy of the strand; determine a reference matrix based on a second copy of the strand, where each column of the reference matrix repeats the second copy of the strand; determine a correlation matrix based on a comparison of corresponding bases or base pairs in the convolutional matrix and the reference matrix; determine a most likely path through the correlation matrix corresponding to offset values between the first copy of the strand and the second copy of the strand; correct base pair alignment of the at least two copies of the strand based on the most likely path. Such a sequencer may be part of a decoder used in a DNA storage device or system.

Implementations may include one or more of the following features. Determining the most likely path through the correlation matrix may include applying a Viterbi algorithm to traverse the correlation matrix. The correlation matrix may include: rows corresponding to a sequence of positions along a length of the strand; columns corresponding to single base pair shifts in relative positions of the first copy of the strand and the second copy of the strand; and matrix values corresponding to an exclusive-or comparison of corresponding bases or base pairs of the first copy of the strand and the second copy of the strand. Determining the most likely path through the correlation matrix may include: traversing the correlation matrix to determine a series of probabilities of changing from a current column to an adjacent column for each row; and calculating, based on the series of probabilities, a path having a highest likelihood among possible paths. Traversing the correlation matrix may include: traversing the correlation matrix in a first direction across the correlation matrix to determine forward probabilities; and traversing the correlation matrix in an opposite direction across the correlation matrix to determine reverse probabilities. Calculating the path having the highest likelihood among possible paths may use a summation of the forward probabilities and the reverse probabilities. Determining the series of probabilities of changing from a current column to the adjacent column is based on a Toeplitz matrix. The plurality of copies of the strand may include multiple pairs of copies of the strand. The decoder may be further configured to: determine, for each pair of copies of the multiple pairs of copies of the strand, a corresponding convolutional matrix, a corresponding reference matrix, a corresponding correlation matrix, and corresponding most likely path through the corresponding correlation matrix; and compare the corresponding most likely paths from the multiple pairs of copies of the strand to determine errors. Correcting base pair alignment of the at least two copies of the strand to eliminate errors may be based on a consensus from the multiple pairs of copies of the strand. The decoder may be further configured to average, responsive to correcting base pair alignment of the at least two copies of the strand, the at least two copies of the strand to eliminate mutation or erasure errors; and the sequence of bases or base pairs stored as data may be based on the average of the at least two copies of the strand. The system may include a sequencer configured to: receive a plurality of physical copies of the strand; generate, for at least two physical copies of the plurality of physical copies of the strand, current values corresponding to each base pair in the sequence of bases or base pairs for that physical copy of the strand; and store, for each physical copy of the at least two physical copies of the strand, the read data for the at least two copies of the strand based on the current values in a non-transient data storage medium.

Another general aspect includes a method that includes: receiving read data determined from sequencing at least two copies of an strand, where the strand may include a sequence of bases or base pairs; determining a convolutional matrix based on a first copy of the strand, where each column of the convolutional matrix corresponds to a base pair offset of the first copy of the strand; determining a reference matrix based on a second copy of the strand, where each column of the reference matrix repeats the second copy of the strand; determining a correlation matrix based on a comparison of corresponding bases or base pairs in the convolutional matrix and the reference matrix; determining a most likely path through the correlation matrix corresponding to offset values between the first copy of the strand and the second copy of the strand; correcting base pair alignment of the at least two copies of the strand based on the most likely path.

Implementations may include one or more of the following features. Determining the most likely path through the correlation matrix may include applying a Viterbi algorithm to traverse the correlation matrix. The correlation matrix may include: rows corresponding to a sequence of positions along a length of the strand; columns corresponding to single base pair shifts in relative positions of the first copy of the strand and the second copy of the strand; and matrix values corresponding to an exclusive-or comparison of corresponding bases or base pairs of the first copy of the strand and the second copy of the strand. Determining the most likely path through the correlation matrix may include: traversing the correlation matrix to determine a series of probabilities of changing from a current column to an adjacent column for each row; and calculating, based on the series of probabilities, a path having a highest likelihood among possible paths. Traversing the correlation matrix may include: traversing the correlation matrix in a first direction across the correlation matrix to determine forward probabilities; and traversing the correlation matrix in an opposite direction across the correlation matrix to determine reverse probabilities. Calculating the path having the highest likelihood among possible paths may use a summation of the forward probabilities and the reverse probabilities. Determining the series of probabilities of changing from a current column to the adjacent column may be based on a Toeplitz matrix. The at least two copies of the strand may include multiple pairs of copies of the strand. The method may include: determining, for each pair of copies of the multiple pairs of copies of the strand, a corresponding convolutional matrix, a corresponding reference matrix, a corresponding correlation matrix, and corresponding most likely path through the corresponding correlation matrix; and comparing the corresponding most likely paths from the multiple pairs of copies of the strand to determine errors. Correcting base pair alignment of the at least two copies of the strand to eliminate errors may be based on a consensus from the multiple pairs of copies of the strand. The method may include averaging, responsive to correcting base pair alignment of the at least two copies of the strand, the at least two copies of the strand to eliminate mutation or erasure errors, where sequence of bases or base pairs stored as data is based on the average of the at least two copies of the strand.

Still another general aspect includes a system that includes: means for receiving read data determined from sequencing at least two copies of an strand, where the strand may include a sequence of bases or base pairs; means for determining a convolutional matrix based on a first copy of the strand, where each column of the convolutional matrix corresponds to a base pair offset of the first copy of the strand; means for determining a reference matrix based on a second copy of the strand, where each column of the reference matrix repeats the second copy of the strand; means for determining a correlation matrix based on a comparison of corresponding bases or base pairs in the convolutional matrix and the reference matrix; means for determining a most likely path through the correlation matrix corresponding to offset values between the first copy of the strand and the second copy of the strand; means for correcting base pair alignment of the at least two copies of the strand based on the most likely path.

The present disclosure describes various aspects of innovative technology capable of applying correlation matrices and Viterbi algorithms to de novo sequencing of DNA strands and similar genomic (e.g., nucleic acid based) data, or as part of recovering sequences used in DNA-based data storage. The configuration of correlation matrix and Viterbi algorithms provided by the technology may be applicable to a variety of computer systems used to sequence unknown DNA sequences. The configuration may be applied to a variety of DNA synthesis and sequencing technologies to generate DNA sequence data that may be used for further sequence processing. The novel technology described herein includes a number of innovative technical features and advantages over prior solutions, including, but not limited to, improved efficiency based on elimination of insertion and deletion errors prior to applying other data sequencing techniques (such as data correlation) to the insertion/deletion corrected sequence data.

### DETAILED DESCRIPTION

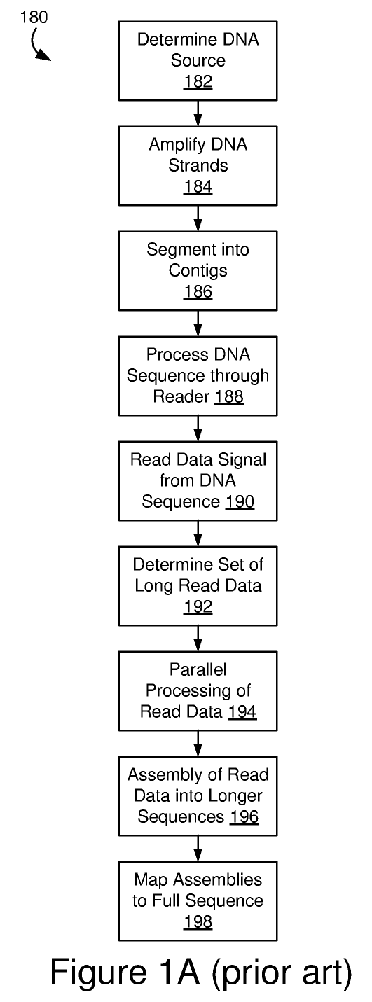

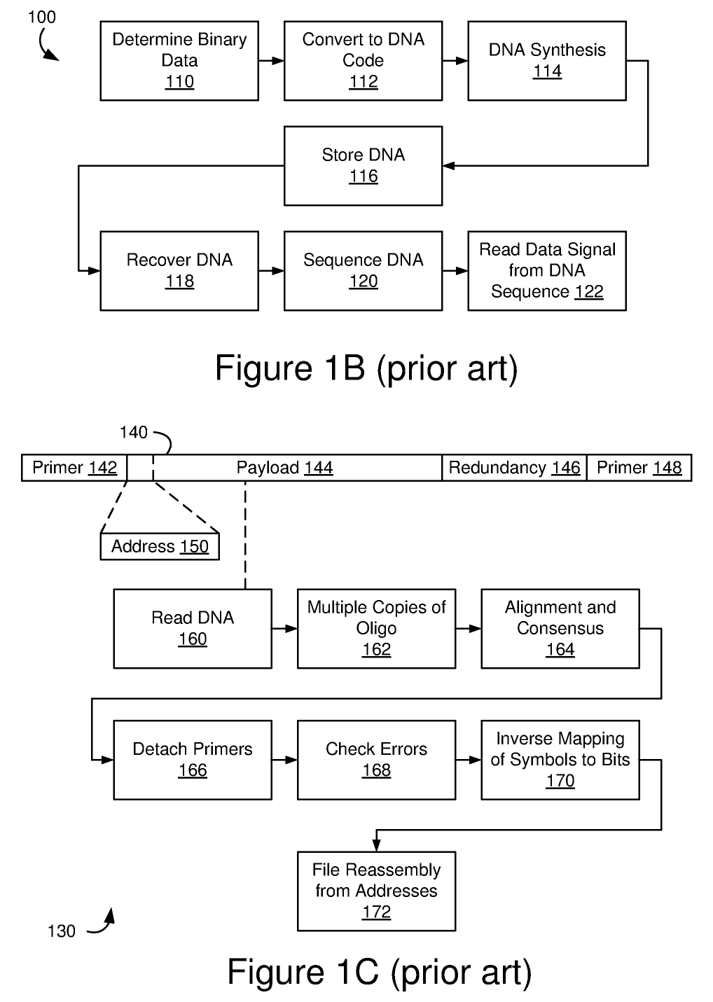

De novo DNA sequencing, including the sequencing of DNA-like molecules such as RNA, may require significant processing power to analyze multiple copies and segments of a larger DNA sequence to rectify transcription and replication errors and determine a “true” copy of the DNA sequence. In an example configuration in FIG. 1A, a method 180 may be used to sequence a DNA sequence to determine and store a true copy of the unknown DNA sequence. While the following descriptions focus on DNA sequences comprised of base pairs, similar techniques may be applied to RNA sequences comprised of bases. The focus on DNA sequencing is not intended to exclude the use of the technology for other nucleic acid-based sequences.

At block 182, the DNA source to be sequenced may be determined. For example, an unknown DNA sample, which may include organic DNA that has not previously been sequenced or synthetic DNA generated for genetic engineering, data storage, or another purpose, may be selected for DNA sequencing.

At block 184, the DNA strands may be amplified. For example, a PCR or similar reaction may be used to replicate the target DNA into multiple copies for sequencing.

At block 186, strands may be segmented into shorter contigs of a target length. For example, long DNA strands representing a larger gene sequence may be divided into a plurality of smaller segments with a target length, such as contigs of n base pairs, where n is a predetermined number, such as 200, 300, 500, etc. Similarly, RNA strands may be divided into a plurality of smaller segments with a target length of n bases.

At block 188, the DNA contigs may be processed through a DNA reader. For example, the short (config) DNA segments may be passed through a nanopore reader to generate read signals from voltages applied across the base pairs in the sequence as they are fed sequentially through the reader.

At block 190, a read data signal may be generated from the DNA sequence. For example, the electrical signal from the sequence reader may generate different voltages representing different base pairs in the sequence and the analog electrical signal may be sampled into a corresponding digital read data signal.

At block 192, a set of long read data segments may be determined. For example, processing a batch of replicated and segmented portions of a longer DNA sequence may generate a set of redundant copies of different contig strands from the replicated DNA strands.

At block 194, the read data for the set of contig strands may be parallel processed through correlation analysis. For example, a set of compute cores may execute parallel correlation analysis from a shared dynamic random access memory (DRAM) to process the set of contig strands.

At block 196, contigs with overlapping sequences may be assembled into longer sequences. For example, the set of contig strands may be processed into longer sequences referred to as assemblies that correspond to larger divisions of the DNA sequence. The number of contigs and assemblies and/or the number of stages of contigs and assemblies to reach the full DNA sequence may be variable based on the length of the DNA sequence and/or the processing resources available for each stage of the processing.

At block 198, assemblies may be mapped to the full DNA sequence. For example, based on overlapping portions of the assemblies, the assemblies may be mapped into the larger DNA sequence to determine a continuous sequence of base pairs corresponding to the length of the DNA sequence. The resulting DNA sequence may be stored to non-volatile memory or otherwise output for use in subsequent sequencing and analysis.

Novel data storage technology is being developed to use synthesized DNA encoded with binary data for long-term data storage. Current approaches may be limited by the time it takes to synthesize and sequence DNA. While the speed of those systems is improving and the density and durability of DNA as a data storage medium is compelling, improvements in de novo sequencing as described herein may assist in the speed of recovery data stored in DNA, particularly when no references for the stored DNA sequence in the synthetic oligos exists. In an example configuration in FIG. 1B, a method 100 may be used to store and recover binary data from synthetic DNA.

At block 110, binary data for storage to the DNA medium may be determined. For example, any conventional computer data source may be targeted for storage in a DNA medium, such as data files, databases, data objects, software code, etc. Due to the high storage density and durability of DNA media, the data targeted for storage may include very large data stores having archival value, such as collections of image, video, scientific data, software, enterprise data, and other archival data.

At block 112, the binary data may be converted to DNA code. For example, a convention computer data object or data file may be encoded according to a DNA symbol index, such as: A or T = 1 and C or G = 0; A=00, T=01, C-10, and G=11; or a more complex DNA symbol index mapping sequences of DNA bases to predetermined binary data patterns. In some configurations, prior to conversion to DNA code, the source data may be encoded according to an oligo-length format that includes addressing and redundancy data for use in recovering and reconstructing the source data during the retrieval process.

At block 114, DNA may be synthesized to embody the DNA code determined at block 112. For example, the DNA code may be used as a template for generating a plurality of synthetic DNA oligos embodying that DNA code using various DNA synthesis techniques. In some configurations, a large data unit is broken into segments matching a payload capacity of the oligo length being used and each segment is synthesized in a corresponding DNA oligo. In some configurations, solid-phase DNA synthesis may be used to create the desired oligos. For example, each desired oligo may be built on a solid support matrix one base at a time to match the desired DNA sequence, such as using phosphoramidite synthesis chemistry in a four-step chain elongation cycle. In some configurations, column-based or microarray-based oligo synthesizers may be used.

At block 116, the DNA medium may be stored. For example, the resulting set of DNA oligos for the data unit may be placed in a fluid or solid carrier medium. The resulting DNA medium of the set of oligos and their carrier may then be stored for any length of time with a high-level of stability (e.g., DNA that is thousands of years old had been successfully sequenced). In some configurations, the DNA medium may include wells of related DNA oligos suspended in carrier fluid or a set of DNA oligos in a solid matrix that can themselves be stored or attached to another object. A set of DNA oligos stored in a binding medium may be referred to as a DNA storage medium for an oligo pool. The DNA oligos in the pool may relate to one or more binary data units comprised of user data (the data to be stored prior to encoding and addition of syntactic data, such as headers, addresses, reference marks, etc.).

At block 118, the DNA oligos may be recovered from the stored medium. For example, the oligos may be separated from the carrier fluid or solid matrix for processing. The resulting set of DNA oligos may be transferred to a new solution for the sequencing process or may be stored in a solution capable of receiving the other polymerase chain reaction (PCR) reagents.

At block 120, the DNA oligos may be sequenced and read into a DNA data signal corresponding to the sequence of bases in the oligo. For example, the set of oligos may be processed through PCR to amplify the number of copies of the oligos from the stored set of oligos. In some configurations, PCR amplification may result in a variable number of copies of each oligo.

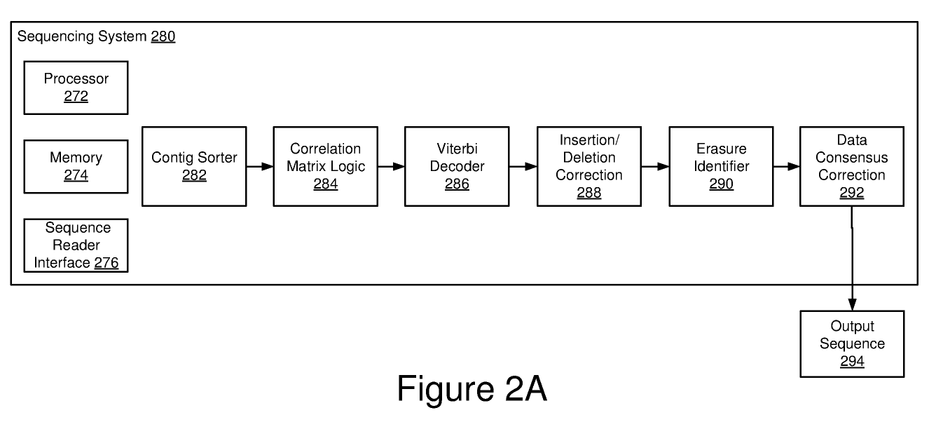

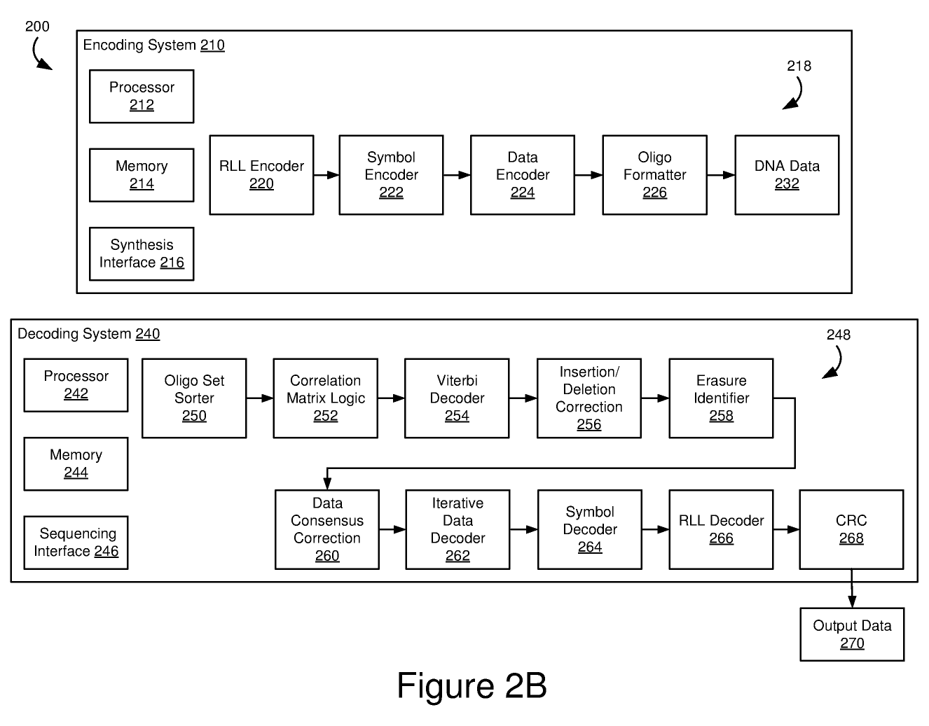

At block 122, a data signal may be read from the sequenced DNA oligos. For example, the sequenced oligos may be passed through a nanopore reader to generate an electrical signal corresponding to the sequence of bases. In some configurations, each oligo may be passed through a nanopore and a voltage across the nanopore may generate a differential signal with magnitudes corresponding to the different resistances of the bases. The analog DNA data signal may then be converted back to digital data based on one or more decoding steps, as further described with regard to a method 130 in FIG. 1C. Improved systems and methods for processing read data from the sequenced oligos to recover the data encoded in the original oligo, including both address/index data and user data, are further described with regard to FIGS. 2-7.

In FIG. 1C, method 130 may be used to convert an analog read signal corresponding to a sequence of DNA bases back to the digital data unit that was the original target of the DNA storage process. In the example shown, the original digital data unit, such as a data file, was broken into data subunits corresponding to a payload size of the oligos and the set of oligos corresponding to the subunits of the data unit may be reassembled into the original data unit. An example oligo format 140, including primers 142 and 148 that may be added to support the PCR amplification and sequencing, may include a payload 144 comprising a subunit of the data unit, a redundancy portion 146 for error correction code (ECC) data for that subunit, and an address portion 150 for determining the sequence of the payloads for reassembling the data block. In some configurations, Reed-Solomon error correction codes may be used to determine the redundancy portion 146 for payload 144.

At block 160, DNA base data signals may be read from the sequenced DNA. For example, the analog signal from the nanopore reader may be conditioned (equalized, filtered, etc.) and converted to a digital data signal for each oligo.

At block 162, multiple copies of the oligos may be determined. Through the amplification process, multiple copies of each oligo may be produced and the decoding system may determine groups of the same oligo to process together.

At block 164, each group of the same oligo may be aligned and consensus across the multiple copies may be determined. For example, a group of four copies may be aligned based on their primers and each base position along the set of base values may have a consensus algorithm applied to determine a most likely version of the oligo for further processing, such as, where 3 out of 4 agree, that value is used.

At block 166, the primers may be detached. For example, primers 142 and 148 may be removed from the set of data corresponding to payload data 144, redundancy data 146, and address 150.

At block 168, error checking may be performed on the resulting data set. For example, ECC processing of payload 144 based on redundancy data 146 may allow errors in the resulting consensus data set for the oligo to be corrected. The number of correctable errors may depend on the ECC code used. ECC codes may have difficulty correcting errors created by insertions or deletions (resulting in shifts of all following base values). The size of the oligo payload 144 and portion allocated to redundancy data 146 may determine and limit the correctable errors and efficiency of the data format.

At block 170, the bases or base symbols may be inversely mapped back to the original bit data. For example, the symbol encoding scheme used to generate the DNA code may be reversed to determine corresponding sequences of bit data.

At block 172, a file or similar data unit may be reassembled from the bit data corresponding to the set of oligos. For example, address 150 from each oligo payload may be used to order the decoded bit data and reassemble the original file or other data unit.

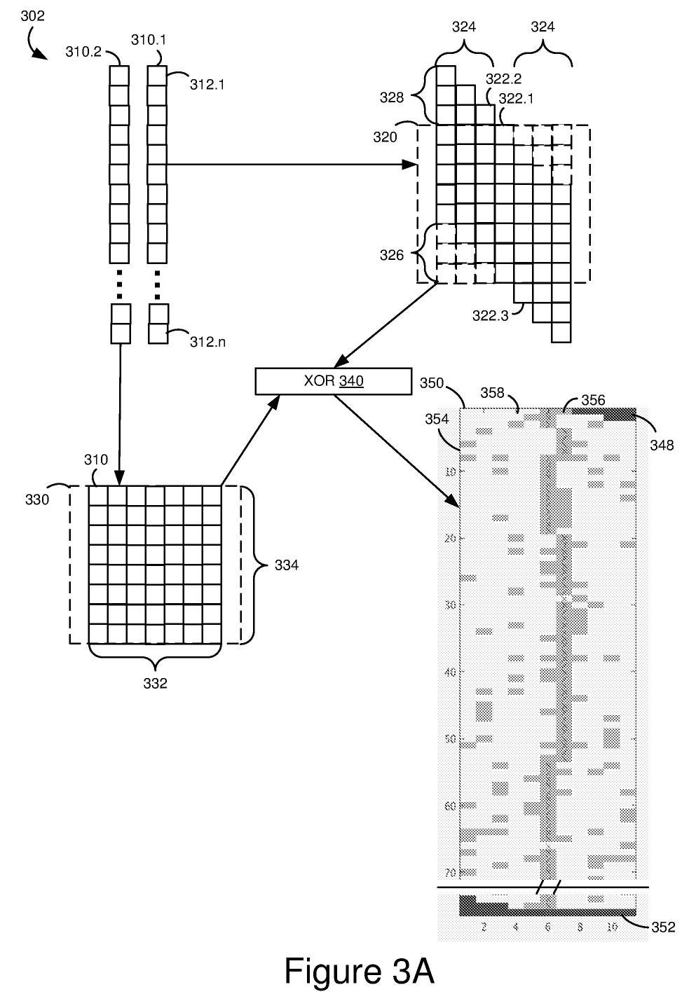

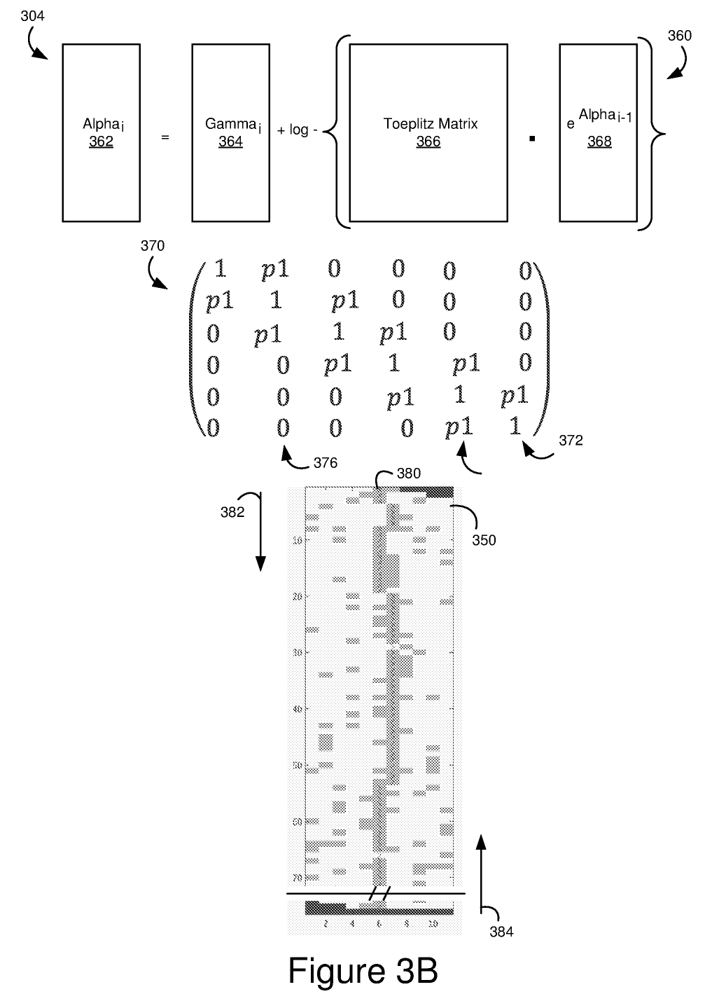

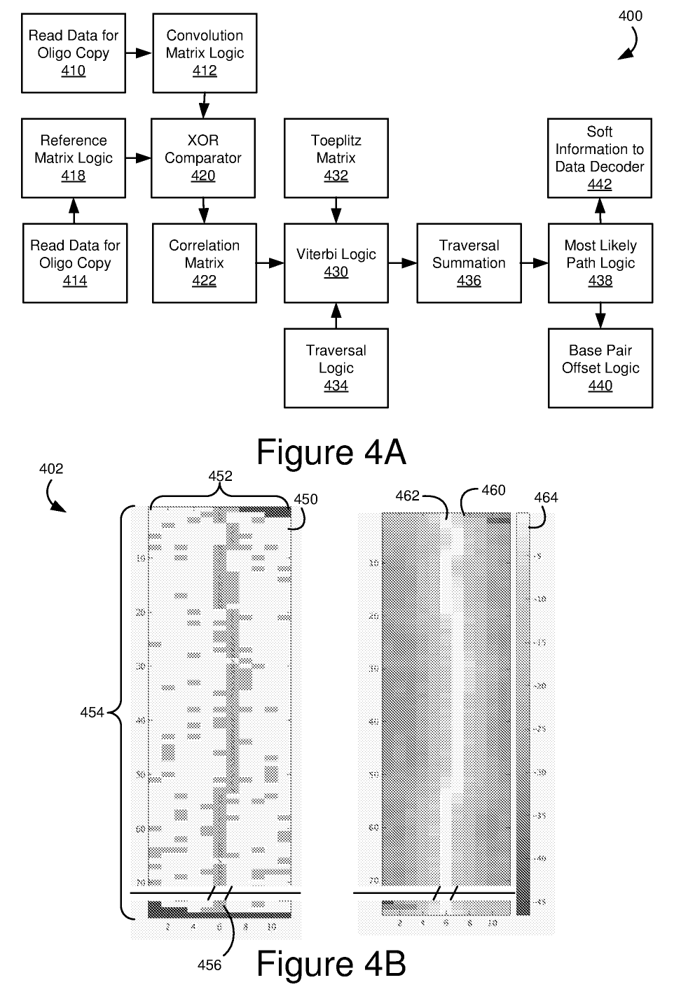

FIG. 2A shows an improved DNA sequencing system 280 and, more specifically, an improved sequencing system using Viterbi processing of a correlation matrix to accelerate one or more stages of sequencing, correcting, and mapping shorter contig strands to assemblies and to full DNA sequences. Sequencing system 280, sometime referred to as a sequencer, may include a processor 272, a memory 274, and a sequence reader interface 276. For example, sequencing system 280 may be part of a computer or storage system or device configured to receive DNA read data, such as a set of contig strand read data, and process it to remove insertion and deletion errors between copies of the same contig strand. In some configurations, sequencing system 280 may be applied at one or more stages in mapping contig strands to assembly strands to full sequences, in as many stages as may be used for sequentially constructing larger and larger sequences. FIG. 2A describes an example configuration for de novo DNA synthesis and FIG. 2B (along with FIGS. 3A-3B) provide a more detailed example applied to de novo sequencing of an unknown synthetic oligo for recovery of DNA storage. The system of FIGS. 4A-4B may be applied to strands or oligos for de novo sequencing or DNA data storage.

Processor 272 may include any type of conventional processor or microprocessor that interprets and executes instructions. In some configurations, processor 272 may include a plurality of processors or processor cores configured to operate alone or in combination to execute one or more functions or sets of instructions described with regard to the other components of sequencing system 280. Memory 274 may include a random-access memory (RAM) or another type of dynamic storage device that stores information and instructions for execution by processor 272 and/or a read only memory (ROM) or another type of static storage device that stores static information and instructions for use by processor 272. In some configurations, one or more components of sequencing system 280 may be embodied in specialized logic and memory circuits configured for the functions described for sequencing system 280 and may incorporate or operating in conjunction with processor 272 and memory 274. For example, one or more encoders, formatters, and/or insertion functions may be embodied in a specialized circuit, such as a system on a chip (SOC), field programmable gate array (FPGA), application specific integrated circuit (ASIC), or similar circuit configuration. Sequencing system 280 may also include any number of input/output devices and/or interfaces. Input devices may include one or more conventional mechanisms that permit an operator to input information to sequencing system 280, such as a keyboard, a mouse, a pen, voice recognition and/or biometric mechanisms, etc. Output devices may include one or more conventional mechanisms that output information to the operator, such as a display, a printer, a speaker, etc. Interfaces may include any transceiver-like mechanism that enables sequencing system 280 to communicate with other devices and/or systems. For example, sequence reader interface 276 may include a connection to an interface bus (e.g., peripheral component interface express (PCIe) bus) or network for communicating the read data from a DNA sequence reader, such as a nanopore reader, to sequencing system 280. In some configurations, sequence reader interface 276 may include a network connection using internet or similar communication protocols to receive a conventional data file listing of the base sequences and/or digital values corresponding to the base pair read signals from the reader. In some configurations, the base sequence read data for the set of contigs or other strands may be stored to conventional removable media, such as a universal serial bus (USB) drive or flash memory card, and transferred from sequencing reader to sequencing system 280 using the removable media.

In some configurations, contig set sorter 282 may include logic to sort a received group of strand read data into sets of copies. For example, the DNA amplification process may result in multiple copies of some or all contig strands and contig set sorter 282 may sort the strand data sequences into like sequences.

In some configurations, correlation matrix logic 284 may include logic for comparing two or more copies of a strand as a first stage to determining insertions and deletions. For example, following synthesis and identification of multiple copies of a strand, insertion and deletion errors would have different locations for different copies and those insertions/deletions. A correlation matrix may be constructed using two copies of the strand. A first copy may be used to construct a convolutional matrix and a second copy may be used to construct a reference matrix. An exclusive-or comparison of the two matrices may yield a correlation matrix for the two copies of the strand.

In some configurations, Viterbi decoder 286 may include logic for determining a most likely path through the correlation matrix from correlation matrix logic 284. For example, following generation of the correlation matrix, a Viterbi algorithm may be used to traverse the matrix and probabilistically determine the most likely correct data in each row of the correlation matrix. The most likely path from Viterbi decoder 286 may indicate by column shifts the most likely positions of insertion and deletion errors.

In some configurations, insertion/deletion correction 288 includes logic for selectively correcting the insertion and deletion errors in a strand, where possible. For example, insertion/deletion correction 288 may use the output from Viterbi decoder 286 to determine correctable insertion/deletion errors. For example, where an insertion or deletion error has occurred, the position of subsequent segments may be corrected for the preceding shift in base pair positions to align the symbols in segments without insertions/deletions with their expected positions in the strand. In some configurations, Viterbi decoder 286 may enable insertion/deletion correction 288 to specifically identify likely locations of insertion and/or deletion errors at the base pair level based on the offsets used in the correlation matrix.

In some configurations, erasure identifier 290 may flag segments of base pairs in the sequence as erasures in need of further error correction, such as correlation analysis. For example, deletions corrected by insertion/deletion correction 288 may result in a placeholder value for data consensus correction 292.

In some configurations, data consensus correction 292 may include logic for using comparison of multiple preprocessed copies that have had their data positions recovered to reduce the number of erasure errors and/or resolve inconclusive corrections. For example, correlation analysis across more than two copies of a strand may allow statistical methods and soft information values to be compared to a correction threshold for deleting inserted base pairs, inserting padding or placeholder base pairs (which may be identified as erasures by erasure identifier 290), and/or correcting mutation errors that appear in a minority of copies. The correction threshold may depend on the number of copies being cross-correlated, decoder signal-to-noise ratio (SNR), size of the insertion/deletion event, and/or a reliability value of the statistical method.

The resulting output sequence 294 for the strand may be output by sequencing system 280. For example, output sequence 294 may include a conventional binary data file corresponding to the strand sequence that may be stored in a non-volatile storage medium, displayed, or transferred to another system for further processing and use. In some configurations, output sequence 294 may be an input to a next stage of sequencing to combine shorter contig strands into assemblies and/or various stages of assemblies into a full DNA sequence.

FIG. 2B shows an improved DNA storage system 200 and, more specifically, an improved encoding system 210 and decoding system 240 for using multistage error correction to correct for insertion/deletion errors based on Viterbi processing of a correlation matrix before applying correlation and/or other error correction techniques to address mutation and/or erasure errors. In some configurations, encoding system 210 may be part of a first computer or storage system or device used for determining target binary data, such as a conventional binary data unit, and converting it to a DNA base sequence for synthesis into DNA for storage and decoding system 240 may be part of a second computer or storage system or device used for receiving the data signal corresponding to the base sequence read from the DNA.

Encoding system 210 may include a processor 212, a memory 214, and a synthesis system interface 216. For example, encoding system 210 may be part of a computer or storage system or device configured to receive or access conventional computer data, such as data stored as binary files, blocks, data objects, databases, etc., and map that data to a sequence of DNA bases for synthesis into DNA storage units, such as a set of DNA oligos. Processor 212 may include any type of conventional processor or microprocessor that interprets and executes instructions. In some configurations, processor 212 may include a plurality of processors or processor cores configured to operate alone or in combination to execute one or more functions or sets of instructions described with regard to the other components of encoding system 210. Memory 214 may include a random-access memory (RAM) or another type of dynamic storage device that stores information and instructions for execution by processor 212 and/or a read only memory (ROM) or another type of static storage device that stores static information and instructions for use by processor 212. In some configurations, one or more components of encoding system 210 may be embodied in specialized logic and memory circuits configured for the functions described for encoding system 210 and may incorporate or operating in conjunction with processor 212 and memory 214. For example, one or more encoders, formatters, and/or insertion functions may be embodied in a specialized circuit, such as a system on a chip (SOC), field programmable gate array (FPGA), application specific integrated circuit (ASIC), or similar circuit configuration. Encoding system 210 may also include any number of input/output devices and/or interfaces. Input devices may include one or more conventional mechanisms that permit an operator to input information to encoding system 210, such as a keyboard, a mouse, a pen, voice recognition and/or biometric mechanisms, etc. Output devices may include one or more conventional mechanisms that output information to the operator, such as a display, a printer, a speaker, etc. Interfaces may include any transceiver-like mechanism that enables encoding system 210 to communicate with other devices and/or systems. For example, synthesis interface 216 may include a connection to an interface bus (e.g., peripheral component interface express (PCIe) bus) or network for communicating the DNA base sequences for storing the data to a DNA synthesis system. In some configurations, synthesis system interface 216 may include a network connection using internet or similar communication protocols to send a conventional data file listing the DNA base sequences for synthesis, such as the desired sequence of bases for each oligo to be synthesized, to the DNA synthesis system. In some configurations, the DNA base sequence listing may be stored to conventional removable media, such as a universal serial bus (USB) drive or flash memory card, and transferred from encoding system 210 to the DNA synthesis system using the removable media.

In some configurations, a series of processing components 218 may be used to process the target binary data, such as a target data file or other data unit, into the DNA base sequence listing for output to the synthesis system. For example, processing components 218 may be embodied in encoder software and/or hardware encoder circuits. In some configurations, processing components 218 may be embodied in one or more software modules stored in memory 214 for execution by processor 212. Note that the series of processing components 218 are examples and different configurations and ordering of components may be possible without materially changing the operation of processing components 218. For example, in an alternate configuration, additional data processing, such as a data randomizer to whiten the input data sequence, may be used to preprocess the data before encoding. In another configuration, user data from a target data unit may be divided across a set of oligos according to oligo payload size or other data formatting prior to applying any encoding or sync marks may be added after ECC encoding. Other variations are possible.

In some configurations, processing the target data may begin with a run length limited (RLL) encoder 220. RLL encoder 220 may modulate the length of stretches in the input data. RLL encoder 220 may employ a line coding technique that processes arbitrary data with bandwidth limits. Specifically, RLL encoder 220 may bound the length of stretches of repeated bits or specific repeating bit patterns so that the stretches are not too long or too short. By modulating the data, RLL encoder 220 can reduce problematic data sequences that could create additional errors in subsequent encoding and/or DNA synthesis or sequencing. In some configurations, RLL encoder 220 or a similar data modulation component may be configured to modulate the input data to assure that data patterns used for syntax references do not appear elsewhere in the user data encoded in the oligo.

In some configurations, symbol encoder 222 may include logic for converting binary data into symbols based on the four DNA bases (adenine (A), cytosine (C), guanine (G), and thymine (T)). In some configurations, symbol encoder 222 may encode each bit as a single base pair, such as 1 mapping to A or T and 0 mapping to G or C. In some configurations, symbol encoder 222 may encode two-bit symbols into single bases, such as 11 mapping to A, 00 mapping to T, 01 mapping to G, and 10 mapping to C. More complex symbol mapping can be achieved based on multi-base symbols mapping to correspondingly longer sequences of bit data. For example, a two-base symbol may correspond to 16 states for mapping four-bit symbols or a four-base symbol may map the 256 states of byte symbols. Multi-base pair symbols could be preferable from an oligo synthesis point of view. For example, synthesis could be done not on base pairs but on lager blocks, like ‘bytes’ correlating to a symbol size, which are prepared and cleaned up earlier (e.g., pre-synthesized) in the synthesis process. This may reduce the amount of synthesis errors. From an encoder/decoder point of view, these physically larger blocks could be treated as symbols or a set of smaller symbols.

In some configurations, data encoder 224 may include logic for encoding the user data unit using one or more error correction schemes and may encode user data units across multiple oligos. For example, encoding system 210 may use low-density parity check (LDPC) codes constructed for the oligo size and/or larger codewords than can be written to a single oligo. In some configurations, data across multiple oligos may be aggregated to form the desired codewords. Similarly, parity or similar redundancy data may not need to be written to each oligo and may instead be written to only a portion of the oligos or written to separate parity oligos that are added to the oligo set for the target data unit. In some configurations, ECC encoding may then be nested for increasingly aggregated sets of oligos, where each level of the nested ECC corresponds to increasingly larger codewords comprised of more oligos. Encoding system 210 may include one or more oligo aggregators and corresponding iterative encoders. For example, single level ECC encoding may use first level oligo aggregator and first level iterative encoder for codewords of 200-400 oligos. A two-level encoding scheme would use first and second level oligo aggregators for and corresponding first and second level iterative encoders, such as for 200 oligo codewords at the first level and 4000 oligo codewords at the second level.

Data encoder 224 may append one or more parity bits to the sets of codeword data for later detection whether certain errors occur during data reading process. For instance, an additional binary bit (a parity bit) may be added to a string of binary bits that are moved together to ensure that the total number of “1”s in the string is even or odd. The parity bits may thus exist in two different types, an even parity in which a parity bit value is set to make the total number of “1”s in the string of bits (including the parity bit) to be an even number, and an odd parity in which a parity bit is set to make the total number of “1”s in the string of bits (including the parity bit) to be an odd number. In some examples, data encoder 224 may implement a linear error correcting code, such as LDPC codes or other turbo codes, to generate codewords that may be written to and more reliably recovered from the DNA medium. In some configurations, resulting parity or similar redundancy data may be stored in parity oligos designated to receive the redundancy data for the set of oligos that make up the codeword data. This additional parity data may be encoded using RLL encoder 220, symbol encoder 222, and/or oligo formatter 226.

In some configurations, oligo formatter 226 may include logic for allocating portions of the target data unit to a set of oligos. For example, oligo formatter 226 may be configured for a predetermined payload size for each oligo and select a series of symbols corresponding to the payload size for each oligo in the set. In some configurations, the payload size may be determined based on an oligo size used by the synthesis system and any portions of the total length of the oligo that are allocated to redundancy data, address data, reference mark data, or other data formatting constraints. For example, for a 150 base pair oligo using two-base symbols may include an eight-base addressing scheme and six four-base sync marks, resulting in 118 base pairs of the target data allocated to each oligo. In some configurations, oligo formatter 226 may insert a unique oligo address or oligo index for each oligo in the set, such as at the beginning or end of the data payload. The oligo address may allow the encoding and decoding systems to identify the data unit and relative position of the symbols in a particular oligo relative to the other oligos that contribute data to that data unit. For example, decoding system 240 may use position information corresponding to the oligo addresses to reassemble the data unit from a set of oligos in an oligo pool containing one or more data units.

The resulting DNA base pair sequence corresponding to the encoded target data unit may be output from processing components 218 as DNA data 232. For example, the base pair sequences for each oligo in the set of oligos corresponding to the target data unit may be stored as sequence listings for transfer to the synthesis system. In some configurations, the base pair sequences may include the encoded data unit data formatted for each oligo, including address, sync mark, and redundancy data added to the user data for the data unit. The set of oligos may include a plurality of first level codeword sets and their corresponding parity oligos and, in some configurations, nested groups of first level codeword sets, second level codeword sets, and so on for as many levels as the particular recovery configuration supports.

Decoding system 240 may include a processor 242, a memory 244, and a sequencing system interface 246. For example, decoding system 240 may be part of a computer or storage system or device configured to receive or access analog and/or digital signal read data from reading sequenced DNA, such as the data signals associated with a set of oligos that have been amplified, sequenced, and read from stored DNA media. Processor 242 may include any type of conventional processor or microprocessor that interprets and executes instructions. In some configurations, processor 242 may include a plurality of processors or processor cores configured to operate alone or in combination to execute one or more functions or sets of instructions described with regard to the other components of encoding system 210. Memory 244 may include a random-access memory (RAM) or another type of dynamic storage device that stores information and instructions for execution by processor 242 and/or a read only memory (ROM) or another type of static storage device that stores static information and instructions for use by processor 242. In some configurations, one or more components of encoding system 210 may be embodied in specialized logic and memory circuits configured for the functions described for encoding system 210 and may incorporate or operating in conjunction with processor 212 and memory 214. For example, one or more encoders, formatters, and/or insertion functions may be embodied in a specialized circuit, such as a system on a chip (SOC), field programmable gate array (FPGA), application specific integrated circuit (ASIC), or similar circuit configuration. Decoding system 240 may also include any number of input/output devices and/or interfaces. Input devices may include one or more conventional mechanisms that permit an operator to input information to decoding system 240, such as a keyboard, a mouse, a pen, voice recognition and/or biometric mechanisms, etc. Output devices may include one or more conventional mechanisms that output information to the operator, such as a display, a printer, a speaker, etc. Interfaces may include any transceiver-like mechanism that enables decoding system 240 to communicate with other devices and/or systems. For example, sequencing system interface 246 may include a connection to an interface bus (e.g., peripheral component interface express (PCIe) bus) or network for receiving analog or digital representations of the DNA sequences from a DNA sequencing system. In some configurations, sequencing system interface 246 may include a network connection using internet or similar communication protocols to receive a conventional data file listing the DNA base sequences and/or corresponding digital sample values generated by analog-to-digital sampling from the sequencing read signal of the DNA sequencing system. In some configurations, the DNA base sequence listing may be stored to conventional removable media, such as a universal serial bus (USB) drive or flash memory card, and transferred to encoding system 240 from the DNA sequencing system using the removable media.

In some configurations, a series of processing components 248 may be used to process the read data, such as a read data file or read data signal from a DNA sequencing system, to output a conventional binary data unit, such as a computer file, data block, or data object. For example, processing components 248 may be embodied in decoder software and/or hardware decoder circuits. In some configurations, processing components 248 may be embodied in one or more software modules stored in memory 244 for execution by processor 242. Note that the series of processing components 248 are examples and different configurations and ordering of components may be possible without materially changing the operation of processing components 248. For example, in an alternate configuration, additional data processing for reversing modulation or other processing from encoding system 216 and/or reassembly of decoded oligo data into larger user data units may be included. Other variations are possible.

Decoding system 240 may use a first stage of error correction targeting the elimination of insertion and deletion errors (which create shifts in all following base pairs in a sequence), followed by ECC error correction to address mutation or erasure errors. DNA media and sequencing face three main types of errors: deletion, insertion, and mutation. Mutation errors are most similar to the traditional errors in data storage and may efficiently be handled using ECC correction. Insertion and deletion errors affect the position of all following bits or symbols and may not be effectively handled by ECC. Therefore, preprocessing the oligo sequences for sequence position shifts and, where possible, correcting those position shifts may contribute to more efficient and reliable data reading. The preprocessing stage may reduce the error rate in the oligo sequences significantly prior to applying ECC correction, enabling more efficient ECC codes and more reliable retrieval of data using a first level of ECC encoding in nested ECC configurations. In some configurations, the preprocessing stage may include oligo set sorter 250, correlation matrix logic 252, Viterbi decoder 254, insertion/deletion correction 256, and erasure identifier 258.

In some configurations, oligo set sorter 250 may include logic to sort a received group of oligo data sequences into sets of copies. For example, the DNA amplification process may result in multiple copies of some or all oligos and oligo set sorter 250 may sort the oligo data sequences into like sequences. Sorting may be based on tagging during the sequencing process, address data (such as address data from the oligo index), and/or statistical analysis of sequences (or samples thereof) to determine repeat copies of each oligo. Note that different copies may include different errors and, at this stage, exact matching of all bases in the sequence may not be the sorting criteria. In this regard, each input oligo may generate a set of one or more oligo copies that correspond to the original input oligo data, but may not be identical copies of that data or of one another, depending on when and how the errors were introduced (thus the need for error correction). A set of oligo copies may be processed together, particularly through the first stage of processing, to determine or generate a best copy of the oligo for ECC processing.

In some configurations, correlation matrix logic 252 may include logic for comparing two or more copies of an oligo as a first stage to determining insertions and deletions. For example, following synthesis and identification of multiple copies of an oligo, insertion and deletion errors would have different locations for different copies and those insertions/deletions. As shown in FIG. 3A, a set of oligo copies, including at least two oligo copies, may result from sequencing and each of those copies may generate corresponding oligo copy read data 310.1 and 310.2. In some configurations, read data 310 may correspond to the sequence of base pairs in the oligo and/or within a payload portion of the oligo. Each set of oligo read data 310 may comprise the base pair sequence 312.1-312.n made up of user data, with or without reference or synchronization marks.

FIG. 3A includes a series of operations 302 to generate and populate a correlation matrix based on the pair of oligo read data copies 310.1 and 310.2. In some configurations, read data 310.1 may be used to generate a convolutional matrix 320. Convolutional matrix 320 may be based on repeating at least a portion of the set of base pairs 312.1-312.n in oligo read data copy 310.1 in each column of the matrix. The center column 322.1 may comprise the full set of base pairs. Each column in either direction, such as column 322.2 and column 322.3) is then offset by one row or base pair in a corresponding direction. For example, each column is shifted one base pair down to the right and one base pair up to the left. Correlation matrix logic 252 may be configured with a number of offset positions 324 in each direction. In the example shown, the number of offset positions 324 equals three in each direction. Number of offset positions 324 may be configured based on a maximum number of insertion or deletion errors the correlation matrix is configured to model. In another example, five offset positions are used in each direction for correlation matrix 350. Where no read values are available due to the offset of the read data, placeholders 326 may be added. These are null values that are not valid portions of convolutional matrix 320 for subsequent calculations. Similarly, read data values 328 that fall outside of convolutional matrix 320 are not represented in the matrix and are shown merely for illustrating the data shift. The resulting convolutional matrix includes rows corresponding to the positions along the oligo and the columns correspond to single base pair offsets or shifts, where right shifts may be created by insertions and left shifts may be created by deletions.

In some configurations, read data 310.2 may be used to generate a reference matrix 330 based on the set of base pairs in another copy of the oligo. Unlike convolutional matrix 320, reference matrix 330 is constructed without offset. Each column 332 repeats the set of base pairs for all positions in the sequence. Each row 334 includes the same base pair values corresponding to that data position in read data 310.2.

An exclusive-or (XOR) operation 340 may then be used to compare each base pair in convolutional matrix 320 with the corresponding matrix position in reference matrix 330. The results of this comparison may populate a correlation matrix 350. The x-axis 352 denotes the columns of the correlation matrix, where the central column from convolutional matrix 320 corresponds to 0 offset and each column to the left or right corresponds to a one base pair offset in that direction. In the example shown, a band of 5 offset positions in each direction is being used, with 6 being the nominal (encoded) position of the base pairs, 1 being a five base pair shift generally corresponding to the direction of deletions and 11 being a five base pair shift generally corresponding to the direction of insertions. The y-axis 354 denotes the rows of the correlation matrix and correspond to base pair positions along the oligo. Medium gray positions, such as block 356, correspond to 0 or base pair matches from the XOR comparison. Light gray positions, such as block 358, correspond to 1 or base pair mismatches from the XOR comparison. Dark gray positions in the corners of the matrix, such as block 348, correspond to the placeholder or null values that are outside the valid positions of the matrix. Correlation matrix 350 may be used to determine the most likely positions of insertions and deletions based on shifts in the matching values by one or more columns in each direction. Note that while convolutional matrix 320 and reference matrix 330 are graphically illustrated with a small number of oligo positions and range of offsets, example correlation matrix 350 is based on a larger oligo size of over 70 base pairs and corresponding base pair positions. Correlation matrix 350 has been truncated, but may be calculated for the full length of the oligo (or data payload portion of the oligo), such as 140 or more base pairs.

In some configurations, Viterbi decoder 254 may include logic for determining a most likely path through the correlation matrix from correlation matrix logic 252. For example, following generation of the correlation matrix, a Viterbi algorithm may be used to traverse the matrix and probabilistically determine the most likely correct data in each row of the correlation matrix. As shown in FIG. 3B, a sequence of operations 304 may use a Viterbi-like algorithm 360 to analyze correlation matrix 350 and identify the most likely path through the sequence of positions. The algorithm applies one or more traverses of the rows of correlation matrix 350 to determine whether the reference position has shifted due to an insertion or deletion. In some configurations, algorithm 360 is a recursive Viterbi algorithm that uses the probability determined for a prior value in the matrix to determine the probability of the next value in the matrix in the direction of the traversal. Alphaᵢ values 362 may include a set of values corresponding to the next row in correlation matrix 350 to be analyzed. Gammaᵢ values 364 may correspond to a set of values for the column shift or offset of the next row. The offset values may be modified by a probabilistic term based on the log of the exponential function of the prior Alpha values 368 (e^{Alpha}ᵢ₋₁) multiplied by a Toeplitz matrix 366. Toeplitz matrix 366 represents the probabilities of moving from column to column (insertions or deletions shifting base pair alignment) and limits the probable movements to a desired range. An example Toeplitz matrix 370 comprises a central diagonal 372 corresponding to no shift and represented by a highest probability value, shown as a 1, but may be more accurately represented as 1-2*p1 such that the total probability for a row adds up to 1. The probability of right shifts (deletions) and left shifts (insertions) may be represented by constant values p1 in the adjacent diagonals 374 to central diagonal 372. In the example shown, the same constant value (p1) is used for both right shifts and left shifts. In an alternate configuration, different constants could be used where the probability of insertions or deletions are determined to be more or less likely than the other. In Toeplitz matrix 370, shifts are limited to a single base pair shift by the 0 constant values 376 beyond adjacent diagonals 374. In some configurations, a low constant value, such as .0001, may be used for values outside of the no shift and shift constants.

The output of Viterbi algorithm 360 may be a set of probabilities for each row, with the highest probability value indicating the most likely shift or offset for that position (or no shift/offset if the highest probability corresponds to the central or prior column). In the example shown, indicator (x) 380 in each row corresponds to the column selected for that row as the most likely shift value based on the highest probability value for that row. In some configurations, Viterbi algorithm 360 may be evaluated in multiple directions and summed or averaged to determine the most likely values for each reference position (row). For example, a first direction 382 may correspond to traversing correlation matrix 350 from the top row (first base pair position in the oligo) to the bottom row (last base pair position in the oligo). A second direction 384 may correspond to traversing correlation matrix 350 in the reverse or opposite direction from the bottom row (last base pair position in the oligo) to the top row (first base pair position in the oligo). The results from the two traversals may then be summed or otherwise evaluated to determine the most likely shift for each reference mark position. In some configurations, the set of probabilities generated by Viterbi algorithm 360 may provide soft information for the corresponding user data positions after the timing of the reference marks is recovered. That soft information may be provided to other decoder functions, such as iterative data decoder 262.

In some configurations, insertion/deletion correction 256 includes logic for selectively correcting the insertion and deletion errors in an oligo, where possible. For example, insertion/deletion correction 256 may use the output from Viterbi decoder 254 to determine correctable insertion/deletion errors. For example, where an insertion or deletion error has occurred, the position of subsequent segments may be corrected for the preceding shift in base pair positions to align the symbols in segments without insertions/deletions with their expected positions in the oligo. In some configurations, Viterbi decoder 254 may enable insertion/deletion correction 256 to specifically identify likely locations of insertion and/or deletion errors at the base pair level based on the offsets used in the correlation matrix.

In some configurations, erasure identifier 258 may flag segments of base pairs in the oligo as erasures in need of ECC error correction. For example, Viterbi decoder 254 and related analysis may determine one or more base pairs between sync marks to be identified as erasures for error correction and/or data consensus correction 260. In some configurations, signal quality, statistical uncertainty, and/or specific thresholds for consensus may cause erasure identifier 258 to identify one or more segments as erasures because the processing by Viterbi decoder 254 and/or data consensus correction 260 is inconclusive. For example, erasure identifier 258 may be configured to output an oligo that has had as many base pairs or symbols as possible positively identified as not containing insertion or deletion errors and identify (and localize) as erasures any base pairs that cannot be determined to be free of insertion or deletion errors prior to performing ECC error correction. Most commonly, placeholders inserted for identified deletions to restore reference mark timing may be identified as erasures. Note that mutation errors in DNA storage may be equivalent to the erasure errors ECC are configured to correct and need not be identified in the preprocessing stage. In some configurations, the insertion/deletion corrected output oligo base pair sequence and related erasure tags and/or soft information from Viterbi decoder 254 may be the output of the preprocessing stage of decoding system 240.

In some configurations, data consensus correction 260 may include logic for using comparison of multiple preprocessed copies that have had their data positions recovered to reduce the number of erasure errors and/or resolve inconclusive corrections. For example, correlation analysis across more than two copies of an oligo may allow statistical methods and soft information values to be compared to a correction threshold for deleting inserted base pairs, inserting padding or placeholder base pairs (which may be identified as erasures by erasure identifier 258), and/or correcting mutation errors that appear in a minority of copies. The correction threshold may depend on the number of copies being cross-correlated, decoder signal-to-noise ratio (SNR), size of the insertion/deletion event, a reliability value of the statistical method, and/or the error correction capabilities of the subsequent ECC processing, including the any nested ECC. In some configurations, data consensus correction 260 may be included as part of the preprocessing stage of decoding system 240.

In some configurations, one or more iterative data decoders 262 may be configured to process the output from the preprocessing stage of decoding system 240. For example, a single “best guess” copy of each unique oligo in a set of oligos for a data unit, including erasure flags and/or soft information, may be passed from preprocessing to ECC decoding. In some configurations, reference marks, address fields, and other formatting data may be removed or ignored by decoding system 240 during ECC processing. Decoding system 240 may use one or more levels of ECC decoding based on aggregating the data from a number of oligos (unique oligos rather than copies of the same oligo). For example, decoding system 240 may use LDPC codes constructed for larger codewords than can be written to or read from a single oligo. Therefore, data across multiple oligos may be aggregated to form the desired codewords. Similarly, parity or similar redundancy data may not be retrieved from each oligo and may instead be read from only a portion of the oligos or from separate parity oligos in the oligo set for the target data unit. In some configurations, ECC decoding may then be nested for increasingly aggregated sets of oligos, where each level of the nested ECC corresponds to increasingly larger codewords comprised of more oligos. Decoding system 240 may include one or more oligo aggregators and corresponding iterative decoders. For example, single level ECC encoding may use first level oligo aggregator and first level iterative encoder for codewords of 200-400 oligos. A two-level encoding scheme would use first and second level oligo aggregators and corresponding first and second level iterative encoders, such as for 200 oligo codewords at the first level and 4000 oligo codewords at the second level.

In some configurations, iterative data decoders 262 may help to ensure that the states at the codeword satisfy the parity constraint by conducting parity error checking to determine whether data has been erased or otherwise lost during data read/write processes. It may check the parity bits appended by data encoder 224 during the data encoding process, and compare them with the base pairs or symbols in the oligo sequences aggregated by the corresponding oligo aggregators. Based on the configuration of data encoder 224 in the data encoding process, each string of recovered bits may be checked to see if the “1”s total to an even or odd number for the even parity or odd parity, respectively. A parity-based post processor may also be employed to correct a specified number of the most likely error events at the output of the Viterbi-like detectors by exploiting the parity information in the coming sequence. In some configurations, iterative data decoder 262 may use soft information received from preprocessing to assist in decode decision-making. When decode decision parameters are met, the codeword may be decoded into a set of decoded base pair and/or symbol values for output or further processing by symbol decoder 264, RLL decoder 266, Cyclic Redundancy Check (CRC) 268, and/or other data postprocessing.

In some configurations, symbol decoder 264 may be configured to convert the DNA base symbols used to encode the bit data back to their bit data representations. For example, symbol decoder 264 may reverse the symbols generated by symbol encoder 222. In some configurations, symbol decoder 264 may receive the error corrected sequences from iterative decoders 262 and output a digital bit stream or bit data representation. For example, symbol decoder may receive a corrected DNA sequence listing for one or more codewords corresponding to the originally stored data unit and process the corrected DNA sequence listing through the symbol-to-bit conversion to generate a bit data sequence.

In some configurations, RLL decoder 266 may decode the run length limited codes encoded by RLL encoder 220 during the data encoding process. In some configurations, the data may go through additional post-processing or formatting to place the digital data in a conventional binary data format. For example, CRC 268 may provide a simple and reliable way to check if the decoded codeword is correct or it is a near codeword. CRC 268 may be implemented as a division of the codeword on a primitive polynomial in some Galois field. The CRC value may be determined for each binary data unit and added by the originating system or encoding system 210. For example, the remainder of the division may be stored in the codeword information for the later CRC check after decoding. CRC 268 may be particularly advantageous for DNA storage, where error rate is high and near codeword detection is more probable. After a successful CRC check, the output data 270 may then be output to a conventional binary data storage medium, network, or device, such as a host computer, network node, etc. for storage, display, and/or use as a convention binary data file or other data unit.

FIG. 4A includes an oligo data processing system 400 using a correlation matrix and Viterbi algorithm to determine data offsets from insertions and deletions. In some configurations, blocks 410-442 may include logic and memory configured to execute the functions of correlation matrix logic 252 and Viterbi decoder 264 in FIG. 2B and associated operations described with regard to FIGS. 3A and 3B. Blocks 410-442 may be embodied in hardware circuits and/or software modules executed using a processor and memory, such as processor 242 and memory 244 in FIG. 2. FIG. 4B includes a series of visualizations 402 of various stages of the data recovery processing.

At block 410, read data for an oligo copy may be received in memory. Convolutional matrix logic 412 may generate a convolutional matrix based on offsetting columns of oligo data by one base pair for a sequence of offset positions in both directions. At block 414, read data for another copy of the same oligo may be received in memory. Reference matrix logic 418 may repeat the sequence of base pairs for each column of a reference matrix to populate that matrix. XOR comparator 420 may include logic to execute a XOR comparison of each corresponding value in the convolutional matrix and the reference matrix to populate correlation matrix 422. For example, XOR comparator 420 may execute XOR logic between the values from each row of the two matrices and return a 0 value for matched base pairs and a 1 value for different base pairs. An example correlation matrix 450 is shown in FIG. 4B, where the light gray areas indicate different base pairs and the medium gray areas indicate matched base pairs. Columns 452 correspond to shifts in base pair position and rows 454 correspond to data positions in the oligo. At this stage, the x-indicators 456 for the most likely path may not yet be determined. As with correlation matrix 350, correlation matrix 450 would include rows corresponding to each base pair being decoded. In the example shown, more than 70 base pairs are depicted, but correlation matrix 450 has been truncated and the number of rows may be 140 or more.

Viterbi logic 430 may operate on correlation matrix 422 to determine probability values for each correlation value in the matrix based on traversing the matrix with a Viterbi algorithm. As described above, Viterbi logic 430 may be configured with a Toeplitz matrix 432 to bias the probabilities for single offset changes from the offset position of the prior base pair position. Viterbi logic 430 may determine a highest probability among the values in each row of correlation matrix 422, as indicated by x-indicators 456 in example correlation matrix 450. In some configurations, Viterbi logic 430 may execute for multiple traverses of correlation matrix 422 based on traversal logic 434. For example, traversal logic 434 may execute Viterbi logic 430 in a first (alpha) direction traversal and a second (beta) direction traversal. The resulting probability values for each matrix position may then be summed or otherwise combined by traversal summation 436. An example output of traversal summation 436 is shown in example probability summation matrix 460 in FIG. 4B. The varying shades of gray correspond to different probability variance from the most likely path 462 (0 values), as indicated by scale 464. Summation matrix 460 has also been truncated.

Most likely path logic 438 may use the probability values determined by Viterbi logic 430 directly or through the summation of multiple traversals of correlation matrix 422 to determine the most likely value (and corresponding offset column) for each row in correlation matrix 422. For example, the highest value in each row of example correlation matrix 450 may be annotated with indicator (x) 456 for the highest values, as determined by most likely path logic 438. The results of most likely path logic 438 may be processed by base pair offset logic 440 to determine the corresponding offsets of the base pairs and the insertion and deletion corrections needed to restore the original oligo data base pair positions. As shown in the example full oligo correlation matrix 470 in FIG. 4B, the reference mark shifts may be determined for the full oligo. Correlation matrix 470 includes the full length 472 of the oligo and the most likely path indicators 474 to guide insertion and deletion correction for the user data. In some configurations, at block 442, the soft information (probability values) determined by Viterbi logic 430 and/or traversal summation 436 may be output to a memory for transfer to an ECC data decoder for decoding the user data.

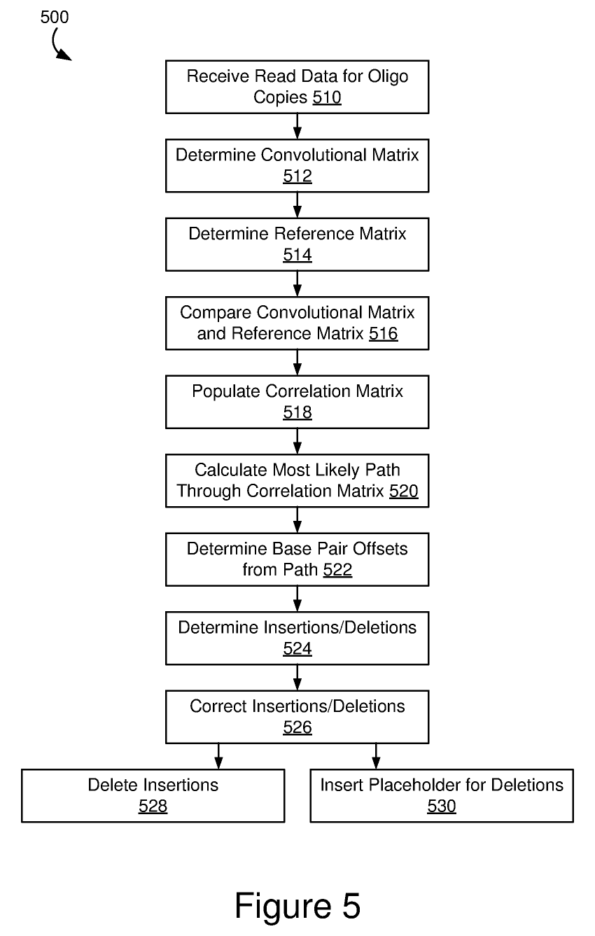

As shown in FIG. 5, the decoder in decoding system 240 may be operated according to an example method of correcting insertions and deletions based on a correlation matrix, i.e., according to the method 500 illustrated by blocks 510-530.

At block 510, read data may be received for at least two oligo copies. For example, the decoder may receive at least two sequences of base pairs corresponding to the same oligo.

At block 512, a convolutional matrix may be determined. For example, the decoder may organize the sequence of base pairs into a column, and then repeat that column for a desired number of offsets in each direction, such as five single base pair offsets.

At block 514, a reference matrix may be determined. For example, the decoder may repeat the base pairs from the second oligo read data for each column of the reference matrix to match the number of columns in the convolutional matrix.

At block 516, the convolutional matrix may be compared to the reference matrix. For example, the decoder may use an exclusive or to compare the base pair values in one matrix with the corresponding matrix position in the other matrix.

At block 518, a correlation matrix may be populated. For example, the decoder may place a corresponding value from the comparison at block 518 in the corresponding matrix position in a correlation matrix of the same size as the convolutional matrix and the reference matrix.

At block 520, a most likely path through the correlation matrix may be determined. For example, the decoder may apply a Viterbi algorithm to traversing the correlation matrix to determine relative probability values in each row of the correlation matrix for possible shifts in base pair positions.

At block 522, base pair offsets may be determined from the most likely path. For example, the decoder may interpret (relative to the most likely value in the prior row) shifts to the column on the left as one direction of offset and shifts to the column on the right as an opposite direction of offset.

At block 524, insertions and deletions may be determined from the offsets. For example, the decoder may evaluate the directions of the offsets as corresponding to sites of insertions or deletions in the oligo copy, such as left offsets corresponding to deletions and right offsets corresponding to insertions.

At block 526, insertions and deletions may be corrected in one or more of the oligo copies. For example, the decoder may include logic to insert placeholder values (which may be marked as erasures) for identified deletions at block 532 and delete base pairs identified as insertions at block 530 to restore the base pair timing of at least one of the oligo copies.

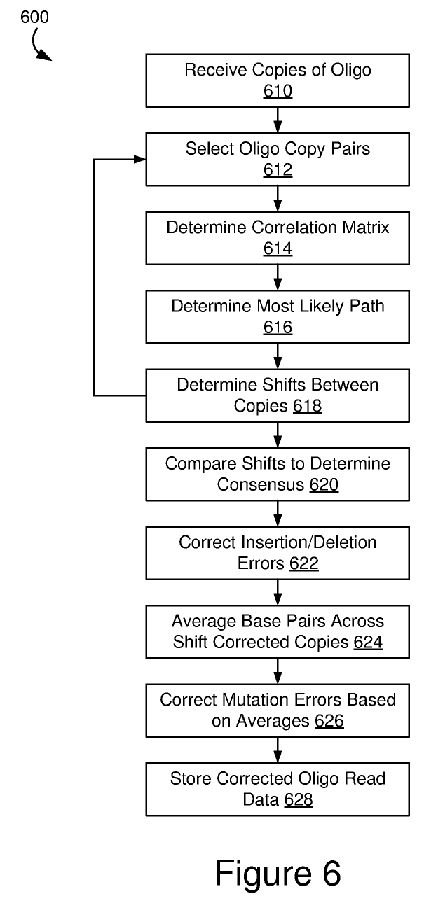

As shown in FIG. 6, the decoder in decoding system 240 may be operated according to an example method of correcting oligo read data based on processing multiple copy pairs of oligo read data, i.e., according to the method 600 illustrated by blocks 610-628.

At block 610, multiple copies of an oligo may be received. For example, the decoder may receive at least four sequences of base pairs corresponding to the same oligo.

At block 612, an oligo copy pair may be selected. For example, the decoder may group the sequences of base pairs into groups of two sets of oligo read data from the same oligo for executing data correction.

At block 614, a correlation matrix may be determined based on comparing the pair of oligo copies. For example, the decoder may use one set of oligo data to generate a convolutional matrix and the other set of oligo data to generate a reference matrix, then compare the two to generate a corresponding correlation matrix.

At block 616, a most likely path may be determined. For example, the decoder may use a Viterbi algorithm to traverse the correlation matrix and determine the most likely path based on the probabilities for each row of the correlation matrix.

At block 618, shifts between the pair of oligo copies may be determined. For example, the decoder may determine and tag the base pair positions with column changes in the most likely path as data shifts between the two copies. In some configurations, method 600 may return to block 612 to select and compare additional pairs. This may include entirely different copies being paired and/or different combinations of pairings among the set of oligo copies. At least four pairs and/or different combinations may be provide a minimum number of shift comparisons for resolving differences.

At block 620, shifts may be compared to determine consensus. For example, the decoder may compare the shifts determined for each pair of copies at block 618 to one another and majority consensus or another consensus algorithm may be used to achieve a desired level of confidence in the accuracy of the shifts in need of correction.

At block 622, insertions and deletion errors may be corrected. For example, the decoder may correct the insertions (by deleting the inserted base pair) and deletions (by inserting a placeholder base pair) in each of the copies based on the consensus analysis.

At block 624, base pairs may be averaged across the shift corrected copies. For example, the decoder may use probability averaging to determine a most likely base pair value among the shift corrected base pair sets of oligo data.

At block 626, mutation or erasure errors may be corrected based on the averages. For example, the decoder may use the averaged probabilities to select the highest probability base pair value for the remaining base pair positions in the shift corrected copies without unanimity. In some configurations, a confidence threshold may be applied to the resulting probability to determine whether the data correction should be made and/or continue to be flagged as an erasure.

At block 628, the corrected oligo read data may be stored. For example, the decoder may store a “best copy” determined by method 600 to a memory location for further use in decoding and recovering the user data stored in the oligo—which may include the use of ECC and reassembly of larger user data units from multiple (different) oligos.

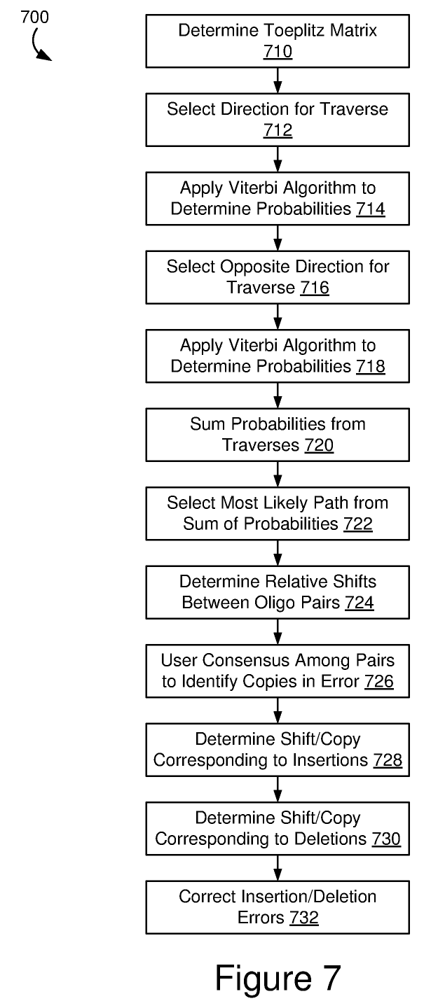

As shown in FIG. 7, the decoder in decoding system 240 and, more specifically, the Viterbi decoder 254, may be operated according to an example method of determining probabilities of reference mark shifts using a Viterbi algorithm, i.e., according to the method 700 illustrated by blocks 710-732. In some configurations, the oligo data processing system 400 of FIG. 4 may be embodied at least in part in a Viterbi decoder and execute method 700. In some configurations, method 700 may operate in the context of method 500 and, more specifically, provide further detail of the operations for block 520 on a correlation matrix populated by that method.

At block 710, a Toeplitz matrix may be determined. For example, the decoder may include a Toeplitz matrix configured to modify probabilities for single base pair offsets from a prior base pair offset position.

At block 712, a direction may be selected for traverse. For example, the decoder may be configured to traverse the correlation matrix in a first or alpha direction, such as from the top row or start of the read data sequence to the bottom row or end of the read data sequence.

At block 714, the Viterbi algorithm may be applied to determine probabilities for each position in the matrix based on the direction of traversal. For example, the decoder may calculate probabilities for each row through the correlation matrix based on the probability values calculated for the prior row in the correlation matrix based on the direction of the traversal.

At block 716, an opposite direction may be selected for traverse. For example, the decoder may be configured to traverse the correlation matrix in a second or beta direction, such as from the bottom row or end of the read data sequence to the top row or start of the read data sequence.

At block 718, the Viterbi algorithm may be applied to determine probabilities for each position in the matrix based on the direction of traversal. For example, the decoder may make the same calculations as at block 714, but in the opposite direction.

At block 720, probabilities from the traverses may be summed. For example, the decoder may combine the results for each position in the matrix across the two traverses in opposite directions, such as by adding, averaging, or variance calculation of the forward probabilities and reverse probabilities.

At block 722, a most likely path may be selected from the sum of probabilities. For example, the decoder may evaluate the summed probabilities in each row of the matrix to determine which probability is the highest among the possible paths through the matrix.

At block 724, relative shifts may be determined between the base pair sets of the pair of oligo copies. For example, the decoder may determine that column shifts from a current column to an adjacent column between one row and the next correspond to an insertion or deletion between the two copies.

At block 726, consensus may be used across multiple pairs to determine which copies are in error. For example, the decoder may user the shifts from multiple pair comparisons to determine the copies that are most likely to be in error.

At block 728, the copies in error and corresponding shifts corresponding to insertions may be determined. For example, the decoder may use the relative shifts from the consensus copies and shifted copies (in error) based on the column shifts in their most likely path to determine the shifts corresponding to insertions based on shift direction.

At block 730, the copies in error and corresponding shifts corresponding to deletions may be determined. For example, the decoder may use the relative shifts from the consensus copies and shifted copies (in error) based on the column shifts in their most likely path to determine the shifts corresponding to deletions based on shift direction.

At block 732, insertions and deletions may be corrected. For example, the decoder may correct the identified insertions and deletions in the corresponding copies in error by deleting insertions and inserting placeholder for deletions (for example, based on consensus identification of the most likely missing base pair).

Technology for improved encoding and decoding of data for DNA storage is described above. In the above description, for purposes of explanation, numerous specific details were set forth. It will be apparent, however, that the disclosed technologies can be practiced without any given subset of these specific details. In other instances, structures and devices are shown in block diagram form. For example, the disclosed technologies are described in some implementations above with reference to particular hardware.

Reference in the specification to “one embodiment” or “an embodiment” means that a particular feature, structure, or characteristic described in connection with the embodiment is included in at least one embodiment or implementation of the disclosed technologies. The appearances of the phrase “in one embodiment” in various places in the specification are not necessarily all referring to the same embodiment or implementation.

Some portions of the detailed descriptions above may be presented in terms of processes and symbolic representations of operations on data bits within a computer memory. A process can generally be considered a self-consistent sequence of operations leading to a result. The operations may involve physical manipulations of physical quantities. These quantities take the form of electrical or magnetic signals capable of being stored, transferred, combined, compared, and otherwise manipulated. These signals may be referred to as being in the form of bits, values, elements, symbols, characters, terms, numbers, or the like.

These and similar terms can be associated with the appropriate physical quantities and can be considered labels applied to these quantities. Unless specifically stated otherwise as apparent from the prior discussion, it is appreciated that throughout the description, discussions utilizing terms for example “processing” or “computing” or “calculating” or “determining” or “displaying” or the like, may refer to the action and processes of a computer system, or similar electronic computing device, that manipulates and transforms data represented as physical (electronic) quantities within the computer system’s registers and memories into other data similarly represented as physical quantities within the computer system memories or registers or other such information storage, transmission or display devices.

The disclosed technologies may also relate to an apparatus for performing the operations herein. This apparatus may be specially constructed for the required purposes, or it may include a general-purpose computer selectively activated or reconfigured by a computer program stored in the computer. Such a computer program may be stored in a computer readable storage medium, for example, but is not limited to, any type of disk including floppy disks, optical disks, CD-ROMs, and magnetic disks, read-only memories (ROMs), random access memories (RAMs), erasable programmable read-only memories (EPROMs), electrically erasable programmable read-only memories (EEPROMs), magnetic or optical cards, flash memories including USB keys with non-volatile memory or any type of media suitable for storing electronic instructions, each coupled to a computer system bus.

The disclosed technologies can take the form of an entire hardware implementation, an entire software implementation or an implementation containing both hardware and software elements. In some implementations, the technology is implemented in software, which includes but is not limited to firmware, resident software, microcode, etc.

Furthermore, the disclosed technologies can take the form of a computer program product accessible from a non-transitory computer-usable or computer-readable medium providing program code for use by or in connection with a computer or any instruction execution system. For the purposes of this description, a computer-usable or computer-readable medium can be any apparatus that can contain, store, communicate, propagate, or transport the program for use by or in connection with the instruction execution system, apparatus, or device.

A computing system or data processing system suitable for storing and/or executing program code will include at least one processor (e.g., a hardware processor) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modems, and Ethernet cards are just a few of the currently available types of network adapters.

The terms storage media, storage device, and data blocks are used interchangeably throughout the present disclosure to refer to the physical media upon which the data is stored.

Finally, the processes and displays presented herein may not be inherently related to any particular computer or other apparatus. Various general-purpose systems may be used with programs in accordance with the teachings herein, or it may prove convenient to construct more specialized apparatus to perform the required method operations. The required structure for a variety of these systems will appear from the description above. In addition, the disclosed technologies were not described with reference to any particular programming language. It will be appreciated that a variety of programming languages may be used to implement the teachings of the technologies as described herein.

The foregoing description of the implementations of the present techniques and technologies has been presented for the purposes of illustration and description. It is not intended to be exhaustive or to limit the present techniques and technologies to the precise form disclosed. Many modifications and variations are possible in light of the above teaching. It is intended that the scope of the present techniques and technologies be limited not by this detailed description. The present techniques and technologies may be implemented in other specific forms without departing from the spirit or essential characteristics thereof. Likewise, the particular naming and division of the modules, routines, features, attributes, methodologies and other aspects are not mandatory or significant, and the mechanisms that implement the present techniques and technologies or its features may have different names, divisions and/or formats. Furthermore, the modules, routines, features, attributes, methodologies and other aspects of the present technology can be implemented as software, hardware, firmware or any combination of the three. Also, wherever a component, an example of which is a module, is implemented as software, the component can be implemented as a standalone program, as part of a larger program, as a plurality of separate programs, as a statically or dynamically linked library, as a kernel loadable module, as a device driver, and/or in every and any other way known now or in the future in computer programming. Additionally, the present techniques and technologies are in no way limited to implementation in any specific programming language, or for any specific operating system or environment. Accordingly, the disclosure of the present techniques and technologies is intended to be illustrative, but not limiting.

1. A system, comprising:
a sequencer configured to:
receive read data determined from sequencing at least two copies of a strand, wherein the strand comprises a sequence of bases or base pairs;
determine a convolutional matrix based on a first copy of the strand, the convolutional matrix comprising a base pair offset introduced to at least a portion of the first copy of the strand;
determine a reference matrix based on a second copy of the strand, wherein the reference matrix repeats at least a portion of the second copy of the strand without introduced base pair offset;
determine a correlation matrix based on a comparison of corresponding bases or base pairs in the convolutional matrix and the reference matrix;
determine a most likely path through the correlation matrix corresponding to offset values between the first copy of the strand and the second copy of the strand;
correct base pair alignment of the at least two copies of the strand based on the determined most likely path.

2. The system of claim 1, wherein determining the most likely path through the correlation matrix comprises applying a Viterbi algorithm to traverse the correlation matrix.

3. The system of claim 1, wherein the correlation matrix comprises:
rows corresponding to a sequence of positions along a length of the strand;
columns corresponding to single base pair shifts in relative positions of the first copy of the strand and the second copy of the strand; and
matrix values corresponding to an exclusive-or comparison of corresponding bases or base pairs of the first copy of the strand and the second copy of the strand.

4. The system of claim 3, wherein determining the most likely path through the correlation matrix comprises:
traversing the correlation matrix to determine a series of probabilities of changing from a current column to an adjacent column for each row; and
calculating, based on the series of probabilities, a path having a highest likelihood among possible paths.

5. The system of claim 4, wherein:
traversing the correlation matrix comprises:
traversing the correlation matrix in a first direction across the correlation matrix to determine forward probabilities; and
traversing the correlation matrix in an opposite direction across the correlation matrix to determine reverse probabilities; and
calculating the path having the highest likelihood among possible paths uses a summation of the forward probabilities and the reverse probabilities.

6. The system of claim 4, wherein determining the series of probabilities of changing from a current column to the adjacent column is based on a Toeplitz matrix.

7. The system of claim 1, wherein:
the plurality of copies of the strand comprise multiple pairs of copies of the strand; and
the sequencer is further configured to:
determine, for each pair of copies of the multiple pairs of copies of the strand:
a corresponding convolutional matrix;
a corresponding reference matrix;
a corresponding correlation matrix; and
corresponding most likely path through the corresponding correlation matrix; and
compare the corresponding most likely paths from the multiple pairs of copies of the strand to determine errors.

8. The system of claim 7, wherein correcting base pair alignment of the at least two copies of the strand to eliminate errors is based on a consensus from the multiple pairs of copies of the strand.

9. The system of claim 8, wherein:
the sequencer is further configured to average, responsive to correcting base pair alignment of the at least two copies of the strand, the at least two copies of the strand to eliminate mutation or erasure errors; and
the sequence of bases or base pairs stored as data is based on the average of the at least two copies of the strand.

10. The system of claim 1, further comprising:
a sequence reader configured to:
receive a plurality of physical copies of the strand;
generate, for at least two physical copies of the plurality of physical copies of the strand, current values corresponding to each base pair in the sequence of bases or base pairs for that physical copy of the strand; and
store, for each physical copy of the at least two physical copies of the strand, the read data for the at least two copies of the strand based on the current values in a non-transient data storage medium.

11. A method comprising:
receiving read data determined from sequencing at least two copies of a strand, wherein the strand comprises a sequence of bases or base pairs;
determining a convolutional matrix based on a first copy of the strand, wherein each column of the convolutional matrix corresponds to a base pair offset of the first copy of the strand;
determining a reference matrix based on a second copy of the strand, wherein each column of the reference matrix repeats the second copy of the strand;
determining a correlation matrix based on a comparison of corresponding bases or base pairs in the convolutional matrix and the reference matrix;
determining a most likely path through the correlation matrix corresponding to offset values between the first copy of the strand and the second copy of the strand;
correcting base pair alignment of the at least two copies of the strand based on the most likely path.

12. The method of claim 11, wherein determining the most likely path through the correlation matrix comprises applying a Viterbi algorithm to traverse the correlation matrix.

13. The method of claim 11, wherein the correlation matrix comprises:
rows corresponding to a sequence of positions along a length of the strand;
columns corresponding to single base pair shifts in relative positions of the first copy of the strand and the second copy of the strand; and
matrix values corresponding to an exclusive-or comparison of corresponding bases or base pairs of the first copy of the strand and the second copy of the strand.

14. The method of claim 13, wherein determining the most likely path through the correlation matrix comprises:
traversing the correlation matrix to determine a series of probabilities of changing from a current column to an adjacent column for each row; and
calculating, based on the series of probabilities, a path having a highest likelihood among possible paths.

15. The method of claim 14, wherein:
traversing the correlation matrix comprises:
traversing the correlation matrix in a first direction across the correlation matrix to determine forward probabilities; and
traversing the correlation matrix in an opposite direction across the correlation matrix to determine reverse probabilities; and
calculating the path having the highest likelihood among possible paths uses a summation of the forward probabilities and the reverse probabilities.

16. The method of claim 14, wherein determining the series of probabilities of changing from a current column to the adjacent column is based on a Toeplitz matrix.

17. The method of claim 11, wherein the at least two copies of the strand comprise multiple pairs of copies of the strand, further comprising:
determining, for each pair of copies of the multiple pairs of copies of the strand:
a corresponding convolutional matrix;
a corresponding reference matrix;
a corresponding correlation matrix; and
corresponding most likely path through the corresponding correlation matrix; and
comparing the corresponding most likely paths from the multiple pairs of copies of the strand to determine errors.

18. The method of claim 17, wherein correcting base pair alignment of the at least two copies of the strand to eliminate errors is based on a consensus from the multiple pairs of copies of the strand.

19. The method of claim 18, further comprising:
averaging, responsive to correcting base pair alignment of the at least two copies of the strand, the at least two copies of the strand to eliminate mutation or erasure errors, wherein the sequence of bases or base pairs stored as data is based on the average of the at least two copies of the strand.

20. A system comprising:
means for receiving read data determined from sequencing at least two copies of a strand, wherein the strand comprises a sequence of bases or base pairs;
means for determining a convolutional matrix comprised of a first copy of the strand, wherein each column of the convolutional matrix corresponds to a base pair offset of the first copy of the strand;
means for determining a reference matrix comprised of a second copy of the strand, wherein each column of the reference matrix repeats the second copy of the strand;
means for determining a correlation matrix based on a comparison of corresponding bases or base pairs in the convolutional matrix and the reference matrix;
means for determining a most likely path through the correlation matrix corresponding to offset values between the first copy of the strand and the second copy of the strand;
means for correcting base pair alignment of the at least two copies of the strand to based on the most likely path.
